(19) United States
(12) Patent Application Publication
(10) Pub. No.: US 2026/0177712 A1
(43) Pub. Date: Jun. 25, 2026

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Tsuyoshi SUZUKI, Tokyo (JP)

(73) Assignee: FUJIFILM Corporation, Tokyo (JP)

(21) Appl. No.: 19/414,323

(22) Filed: Dec. 10, 2025

(30) Foreign Application Priority Data
Dec. 19, 2024 (JP) 2024-223824

(51) Int. Cl. G01T 7/00; A61B 6/03; A61B 6/58; G01T 1/17

(52) U.S. Cl. CPC G01T 7/005; A61B 6/035; A61B 6/585; G01T 1/17

(57) ABSTRACT

An information processing apparatus includes, in a key, character strings related to imaging conditions for a radiation image, includes, in a value, calibration data of a radiation detector provided in a radiographic imaging apparatus that captures the radiation image, and stores the key and the value in association with each other in a storage device.

### CROSS-REFERENCE TO RELATED APPLICATION

This application claims priority from Japanese Patent Application No. 2024-223824, filed on Dec. 19, 2024, the entire disclosure of which is incorporated herein by reference.

### BACKGROUND

### 1. Technical Field

The present disclosure relates to an information processing apparatus, an information processing method, and an information processing program.

### 2. Description of the Related Art

JP2020-038635A discloses a technology of extracting medical information that matches a search condition in a case in which a search request for medical information including a key that is an item of information related to a patient and a value that is content of the key is received.

### SUMMARY

In a radiographic imaging apparatus such as a CT apparatus, correction processing using calibration data of a radiation detector is performed. It is preferable that appropriate calibration data is searched for in a short time from among a plurality of calibration data in a case of capturing a radiation image. For example, in a photon-counting CT apparatus, the number of types of calibration also increases, and thus a large amount of calibration data is present. Therefore, it is preferable that a time for the radiation detector to search for the calibration data is shortened.

The present disclosure has been made in view of the above-described circumstances, and an object of the present disclosure is to provide an information processing apparatus, an information processing method, and an information processing program that can shorten a time for a radiation detector to search for calibration data.

The technology of the present disclosure relates to an information processing apparatus comprising: a processor, in which the information processing apparatus performs a process of storing data in a key-value format, and the processor is configured to: include, in a key, character strings related to imaging conditions for a radiation image; include, in a value, calibration data of a radiation detector provided in a radiographic imaging apparatus that captures the radiation image; and store the key and the value in association with each other in a storage device.

The radiation image may be a CT image, and the radiographic imaging apparatus may be a CT apparatus.

The CT apparatus may include a photon-counting radiation detector.

The character strings related to the imaging conditions may include a character string indicating whether or not a process of reducing the number of projection data items acquired by the CT apparatus is performed.

The character strings related to the imaging conditions may include a character string indicating a creation date and time of the calibration data.

The processor may be configured to: receive a character string to be searched for; and acquire the calibration data stored as the value corresponding to the key that partially matches the received character string from the storage device.

The processor may be configured to: during a period from a timing at which preparation for capturing the radiation image is started to a timing at which capturing the radiation image is started, acquire the calibration data corresponding to the imaging conditions for capturing the radiation image from the storage device.

The technology of the present disclosure relates to an information processing method executed by a processor provided in an information processing apparatus that performs a process of storing data in a key-value format, the information processing method comprising: including, in a key, character strings related to imaging conditions for a radiation image; including, in a value, calibration data of a radiation detector provided in a radiographic imaging apparatus that captures the radiation image; and storing the key and the value in association with each other in a storage device.

The technology of the present disclosure relates to an information processing program causing a processor provided in an information processing apparatus that performs a process of storing data in a key-value format, to execute a process comprising: including, in a key, character strings related to imaging conditions for a radiation image; including, in a value, calibration data of a radiation detector provided in a radiographic imaging apparatus that captures the radiation image; and storing the key and the value in association with each other in a storage device.

According to the present disclosure, the time for the radiation detector to search for the calibration data can be shortened.

### BRIEF DESCRIPTION OF THE DRAWINGS

FIG. 1 is a schematic diagram showing an example of a configuration of a tomographic imaging system.

FIG. 2 is a block diagram showing an example of a hardware configuration of a console.

FIG. 3 is a block diagram showing an example of a functional configuration of the console.

FIG. 4 is a diagram showing character strings included in a key.

FIG. 5 is a diagram showing a case in which a downsampling function is off.

FIG. 6 is a diagram showing a case in which the downsampling function is on.

FIG. 7 is a diagram showing a case in which a downsampling function according to a modification example is on.

FIG. 8 is a flowchart showing an example of a process of storing calibration data.

FIG. 9 is a flowchart showing an example of a process of generating a CT image.

### DETAILED DESCRIPTION

Hereinafter, an embodiment for carrying out the technology of the present disclosure will be described in detail with reference to the drawings.

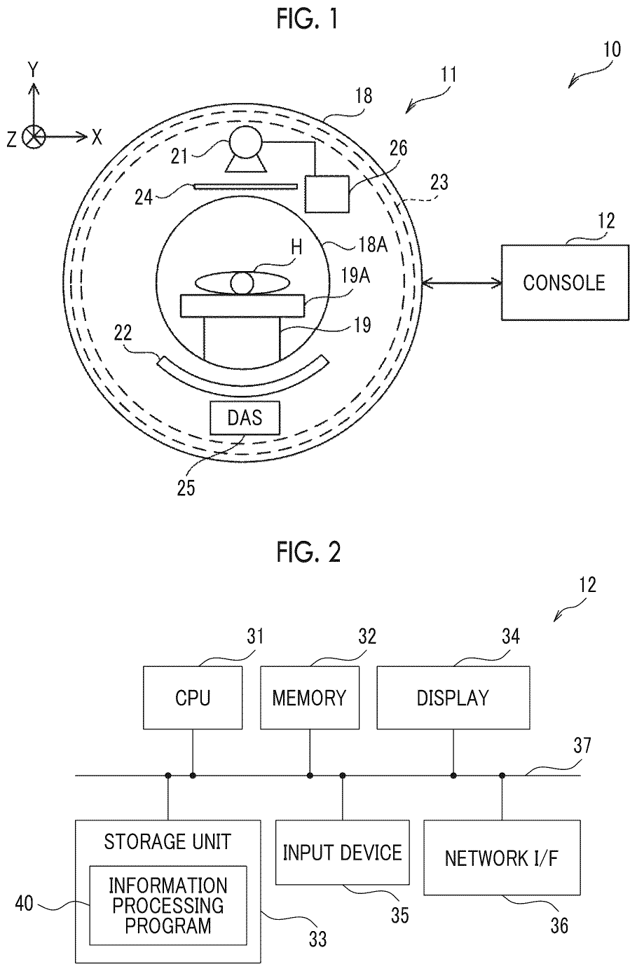

First, a configuration of a tomographic imaging system 10 will be described with reference to FIG. 1. As shown in FIG. 1, the tomographic imaging system 10 according to the present embodiment comprises a CT apparatus 11 and a console 12.

The CT apparatus 11 obtains a tomographic image of a subject H by imaging the subject H using X-rays as an example of radiation. The CT apparatus 11 is an example of a radiographic imaging apparatus that captures a radiation image. The CT image captured by the CT apparatus 11 is an example of a radiation image. The CT apparatus 11 comprises a gantry 18 and an examination table device 19. FIG. 1 is a diagram in which a gantry 18 and the examination table device 19 are viewed from the front side. The examination table device 19 comprises a top plate 19A on which the subject H can be placed in a decubitus posture. In the following description, a longitudinal direction of the top plate 19A will be referred to as a Z axis direction, a lateral direction of the top plate 19A will be referred to as an X axis direction, and a vertical direction will be referred to as a Y axis direction. The top plate 19A can move in the Z axis direction in a state of being kept horizontal. The gantry 18 has an annular shape as a whole, and a circular opening portion 18A having a diameter larger than a width of the top plate 19A is formed at the center thereof. During the imaging, the top plate 19A on which the subject H is placed is moved in the Z axis direction with respect to the gantry 18, to enter the opening portion 18A. The imaging is performed while moving the top plate 19A with respect to the gantry 18.

A radiation source 21, a radiation detector 22, and a frame 23 are disposed inside the gantry 18. The radiation source 21 emits the radiation toward the subject H. The radiation detector 22 detects the radiation transmitted through the subject H. The radiation transmitted through the subject H is attenuated by interaction (for example, absorption and scattering of the radiation) with structures such as organs and bones inside the body of the subject H. The structures each have an attenuation coefficient for the radiation peculiar to the structures, and the radiation transmitted through the structures carries information reflecting the physical properties of the structures. The radiation detector 22 detects the radiation in which physical properties of the structure in the body of the subject H are reflected. The radiation detector 22 has a detection surface in which detection elements are two-dimensionally arranged, and outputs a detection signal for each of the detection elements. For this reason, the radiation detector 22 can detect the radiation at each transmission position transmitted through the structure of the subject H. In addition, the radiation detector 22 has a substantially arc shape in accordance with a curvature of the gantry 18, and the detection surface is also curved. The radiation detector 22 is an example of a photon-counting radiation detector, and is a radiation detector that can count the number of photons of incident X-rays. That is, the CT apparatus 11 is a photon-counting computed tomography (PCCT) apparatus. The radiation detector 22 may be, for example, an energy-integrating type radiation detector that converts X-rays into visible light and then accumulates charges, which are generated by converting the visible light into an electric signal, for a certain time.

The radiation source 21 and the radiation detector 22 are disposed at positions facing each other in the gantry 18 and are rotated around the Z axis while remaining facing each other. The frame 23 has an annular shape and supports the radiation source 21 and the radiation detector 22 in a rotatable manner. During the imaging, the gantry 18 acquires the detection signals by the radiation detector 22 at a plurality of positions in a circumferential direction around the Z axis corresponding to the body axis of the subject H while rotating the radiation source 21 and the radiation detector 22 around the subject H on the top plate 19A. During the imaging, the top plate 19A also moves in the Z axis direction in synchronization with the rotation of the radiation source 21 and the radiation detector 22.

A data acquisition system (DAS) 25 collects the detection signal output by the radiation detector 22, generates output data at each position around the Z axis based on the collected detection signal, and outputs the generated output data to the console 12. In a case in which the subject H is present between the radiation source 21 and the radiation detector 22, this output data is projection data in which the subject H is projected.

An irradiation field limiter 24 (also referred to as a collimator) that limits an irradiation field of the radiation is disposed in front of the radiation source 21 in an irradiation direction. The irradiation field limiter 24 has an irradiation opening of which a contour is defined by a plurality of shielding plates for shielding the radiation, and a size of the irradiation opening can be changed by moving the shielding plates. A voltage is supplied to the radiation source 21 from a high-voltage generator 26. The radiation source 21 and the radiation detector 22 are electrically connected to the frame 23 by a slip ring method, and, for example, power supply, transmission and reception of data, and the like are performed via a slip ring. The connection using the slip ring method allows the radiation source 21 and the radiation detector 22 to perform imaging of a helical scan method in which imaging is performed while rotating in one direction without reversing the rotation direction.

The console 12 controls the radiation source 21 and the radiation detector 22 via a control device (not shown) provided in the gantry 18. The console 12 is an example of an information processing apparatus according to the disclosed technology. The imaging conditions of the CT apparatus 11 are set by the operation from the console 12. The imaging conditions include an irradiation condition of the radiation of the radiation source 21, an imaging range, and the like. The irradiation condition of the radiation includes a tube voltage (unit: kV) to be applied to the radiation source 21, a tube current (unit: mA), and an irradiation time (unit: msec) of the radiation. The imaging range is adjusted, for example, by changing the size of the irradiation opening of the irradiation field limiter 24 in the X-Y plane, and is adjusted by changing a movement range of the top plate 19A in the Z axis direction.

A hardware configuration of the console 12 according to the present embodiment will be described with reference to FIG. 2. Examples of the console 12 include a computer, such as a personal computer or a server computer. As shown in FIG. 2, the console 12 includes a central processing unit (CPU) 31, a memory 32 as a temporary storage area, and a non-volatile storage unit 33. Further, the console 12 includes a display 34 such as a liquid crystal display, an input device 35 such as a keyboard and a mouse, and a network interface (I/F) 36 connected to the CT apparatus 11. The CPU 31, the memory 32, the storage unit 33, the display 34, the input device 35, and the network I/F 36 are connected to a bus 37. The CPU 31 is an example of a processor according to the technology of the present disclosure.

The storage unit 33 is implemented by using a hard disk drive (HDD), a solid state drive (SSD), a flash memory, and the like. An information processing program 40 is stored in the storage unit 33 as a storage medium. The CPU 31 reads out the information processing program 40 from the storage unit 33, loads the read out information processing program 40 into the memory 32, and executes the loaded information processing program 40.

The storage unit 33 stores calibration data of the radiation detector 22. Details of the calibration data will be described later.

In the CT apparatus 11, in a plurality of imaging sequences, it is required to read out the calibration data of the radiation detector 22 within a relatively short time between sequences. Examples of the plurality of imaging sequences include preliminary imaging for determining an imaging range and main imaging in accordance with the imaging range determined in the preliminary imaging. Further, examples of the plurality of imaging sequences include continuous imaging of different parts. In the PCCT apparatus, since the number of types of the calibration data is also relatively large, it is required to search for appropriate calibration data from a large amount of calibration data in a short time. Therefore, the console 12 according to the present embodiment has a function of performing a process of storing the calibration data in a key-value format and searching for the calibration data.

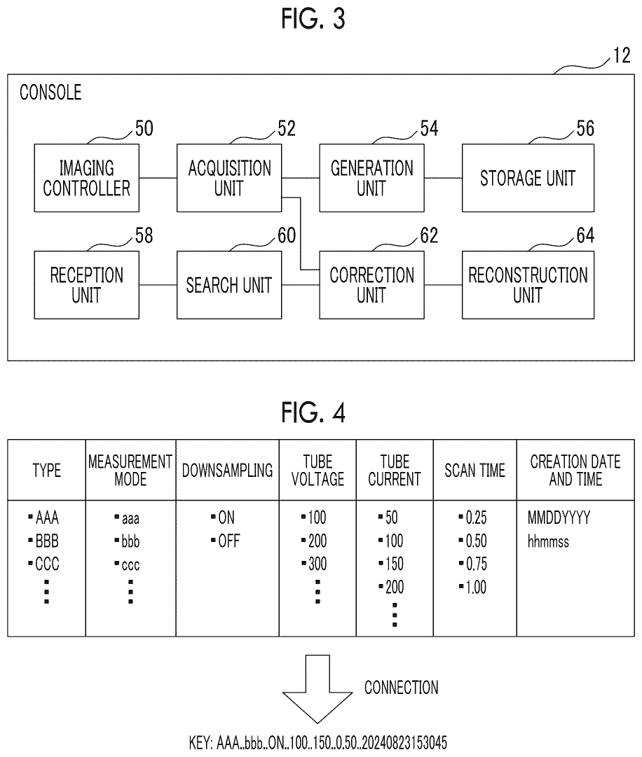

Next, a functional configuration of the console 12 will be described with reference to FIG. 3. As shown in FIG. 3, the console 12 includes an imaging controller 50, an acquisition unit 52, a generation unit 54, a storage unit 56, a reception unit 58, a search unit 60, a correction unit 62, and a reconstruction unit 64. The CPU 31 executes the information processing program 40 to function as the imaging controller 50, the acquisition unit 52, the generation unit 54, the storage unit 56, the reception unit 58, the search unit 60, the correction unit 62, and the reconstruction unit 64.

The imaging controller 50 performs control of capturing the CT image in accordance with the imaging conditions. The imaging controller 50 according to the present embodiment performs the following two types of imaging control. The imaging controller 50 performs, as first imaging control, control of creating the calibration data. For example, the imaging controller 50 performs, as the first imaging control, control of imaging in a state in which the subject H and the examination table device 19 are not present between the radiation source 21 and the radiation detector 22. This control is also referred to as air calibration.

In addition, for example, the imaging controller 50 performs, as the first imaging control, control of imaging in a state in which a phantom is present between the radiation source 21 and the radiation detector 22. This control is also referred to as phantom calibration. The phantom simulates the subject H, and a size, a shape, a material, and the like thereof including a thickness, a length, a width, and the like are known. The phantom is installed by a jig (not shown) or the like.

The imaging controller 50 performs, as second imaging control, control of imaging in a state in which the subject H is present between the radiation source 21 and the radiation detector 22.

The acquisition unit 52 acquires output data (hereinafter, referred to as “first output data”), which is obtained by the first imaging control, from the DAS 25. In a case of the phantom calibration, the first output data is projection data in which the phantom is projected. In addition, the acquisition unit 52 acquires output data (hereinafter, referred to as “second output data”), which is obtained by the second imaging control, from the DAS 25. The second output data is projection data in which the subject H is projected.

The generation unit 54 generates the calibration data for correcting the error included in the output data of the radiation detector 22 based on the first output data acquired by the acquisition unit 52. For example, in the air calibration, in a case in which the first output data does not include the error, it is considered that a projection value based on the first output data is zero. In this case, the generation unit 54 generates the calibration data such that the projection value based on the first output data becomes zero in a case in which the first output data is subtracted for each detection element of the radiation detector 22.

In addition, in the phantom calibration, the size, the shape, the material, and the like of the phantom are known, and the incidence angle of the radiation with respect to the phantom and the radiation dose are also known. Therefore, in the phantom calibration, a theoretical value of the first output data in a case in which the first output data does not include the error can be calculated in advance. In this case, the generation unit 54 generates, as the calibration data, a correction coefficient such that an actually measured value of the first output data matches the theoretical value of the first output data for each detection element of the radiation detector 22.

In addition, the generation unit 54 may generate, as the calibration data, for example, data for correcting the error caused by the deviation of the detection element of the radiation detector 22.

The storage unit 56 includes, in a key, character strings related to the imaging conditions for the CT image in the first imaging control, and includes, in a value, the calibration data of the radiation detector 22 generated based on the first output data obtained in accordance with the imaging conditions. The storage unit 56 stores the key and the value in association with each other in the storage unit 33 as an example of a storage device.

As shown in FIG. 4 as an example, the character strings related to the imaging conditions included in the key according to the present embodiment include a character string indicating each of a type of the calibration data, a measurement mode, and whether the downsampling function is on or off. The type of the calibration data indicates, for example, whether the calibration data is obtained by the air calibration, whether the calibration data is obtained by the phantom calibration, or whether the calibration data is for correcting the error caused by the deviation of the detection element. The measurement mode indicates the number of divisions and a boundary value of the energy band in a case in which the radiation detector 22 counts the photons.

Whether the downsampling function is on or off indicates whether or not a process of reducing the number of projection data items acquired by the CT apparatus 11 is performed in a case in which the CT image for the user to check whether or not the imaging is normally performed during the imaging is generated. In a case in which the CT image of the subject H is being captured, it is preferable that no delay occurs in the process of storing the projection data in the storage unit 33 and the process of generating the CT image for the user to check whether or not the imaging is normally performed. Therefore, in the tomographic imaging system 10 according to the present embodiment, in order to prevent the console 12 from being highly loaded, it is possible to set whether or not the number of projection data items used for generating the CT image for checking whether or not the imaging is normally performed is reduced. This is because the load of various types of correction processing performed on the projection data is relatively high. In a case in which the downsampling function is on, the number of projection data items on which the correction processing is performed is reduced, and thus the console 12 is prevented from being highly loaded.

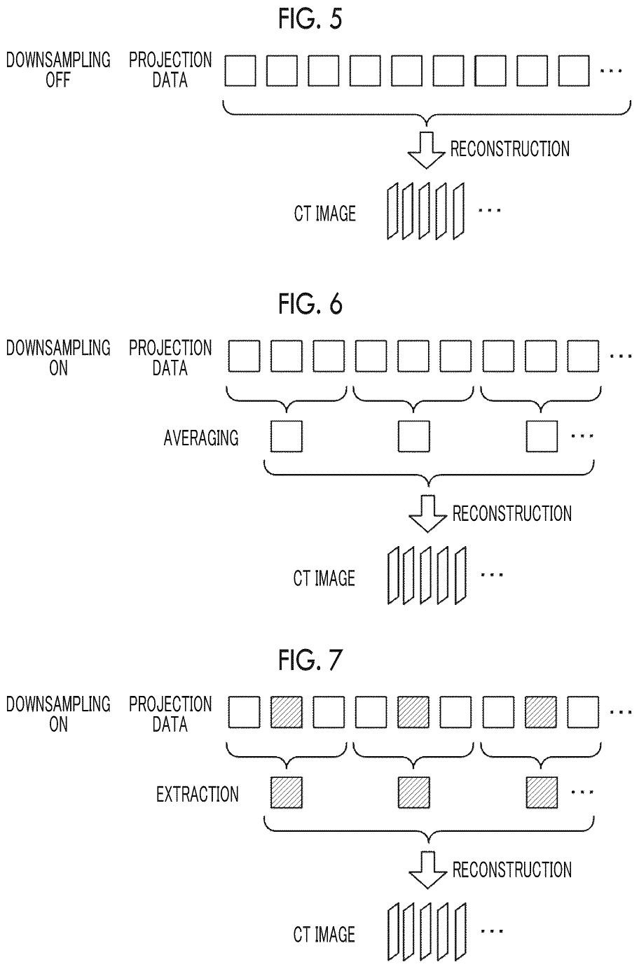

As shown in FIG. 5 as an example, in a case in which the downsampling function is off, the CT image is generated by reconstructing the image based on all the projection data acquired by the CT apparatus 11. On the other hand, as shown in FIG. 6 as an example, in a case in which the downsampling function is on, the process of reducing the number of projection data items acquired by the CT apparatus 11 is performed, and the CT image is generated by reconstructing the image based on the projection data of which the number is reduced. FIG. 6 shows an example in which the number of projection data items is reduced by generating one projection data by averaging the preset number of first projection data items (three items in the example of FIG. 6).

As shown in FIG. 7 as an example, in a case in which the downsampling function is on, the number of projection data items may be reduced by extracting one projection data from the preset number of first projection data items (three items in the example of FIG. 6). FIG. 7 shows an example in which the projection data shaded with diagonal lines is extracted.

In addition, as shown in FIG. 4, the character strings related to the imaging conditions included in the key include a character string indicating each of the tube voltage, the tube current, a scan time, and a creation date and time of the calibration data applied to the radiation source 21. The tube voltage and the tube current indicate numerical values. The scan time indicates a time required for a process in which the radiation source 21 and the radiation detector 22 rotate once around the Z axis. The creation date and time of the calibration data indicates a creation date and time in a preset format. FIG. 4 shows an example in which a format of the creation date and time is “YYYYMMDDhhmmss”. In this case, for example, “Aug. 23, 2024, 15:30:45” is represented by “20240823153045”.

The storage unit 56 generates the key by connecting the character strings indicating the imaging conditions via a delimiter. In FIG. 4, an example is shown in which an underscore (_) is applied as the delimiter.

The reception unit 58 receives the character string to be searched for. For example, the reception unit 58 may receive the character string to be searched for, which is input by the user via the input device 35 based on the imaging conditions in the second imaging control. In addition, for example, the reception unit 58 may receive the character string to be searched for, which is extracted and generated from the imaging conditions in the second imaging control.

The search unit 60 acquires the calibration data in accordance with the imaging conditions of the second imaging control by using the character string received by the reception unit 58. Specifically, the search unit 60 searches for the key that partially matches the character string to be searched for from the keys stored in the storage unit 33. The search unit 60 acquires the calibration data stored as the value corresponding to the searched key from the storage unit 33. For example, in a case in which the character string to be searched for is “AAA_bbb_ON_100_150_0.50_”, the calibration data associated with the key including “AAA_bbb_ON_100_150_0.50_” is acquired.

The search unit 60 may acquire, during a period from a timing at which preparation for capturing the CT image is started to a timing at which capturing the CT image is started, the calibration data in accordance with the imaging conditions for capturing the CT image from the storage unit 33. The period from the timing at which the preparation for capturing is started to the timing at which the capturing is started is, for example, a period from a timing at which capturing the scout image is started to a timing at which the main imaging is started based on the imaging range determined by using the scout image. In addition, the period from the timing at which the preparation for capturing is started to the timing at which the capturing is started is, for example, a period from a timing at which the imaging of a first imaging target part ends (that is, a timing at which the preparation for imaging the second imaging target part is started) to a timing at which the imaging of a second imaging target part is started in a case in which the first imaging target part and the second imaging target part are continuously imaged in this order.

The correction unit 62 corrects the second output data acquired by the acquisition unit 52, using the calibration data acquired by the search unit 60. In a case in which the search unit 60 acquires a plurality of calibration data of different types, the correction unit 62 may correct the second output data using each of the plurality of calibration data. In addition, in a case in which the search unit 60 acquires a plurality of calibration data of the same type, the correction unit 62 may generate one calibration data by averaging the plurality of calibration data.

In addition, in a case in which the search unit 60 acquires a plurality of calibration data of the same type, the correction unit 62 may select one calibration data, which is created most recently, from the plurality of calibration data. In this case, the correction unit 62 may specify the most recently created calibration data from the character string indicating the imaging date and time included in the key. As a result, the calibration data having high accuracy can be acquired even in a case in which the characteristics of the radiation detector 22 change over time. Even in a case in which the CT image is generated by using the projection data acquired in the past, the correction unit 62 can acquire the calibration data created at a date and time closest to the acquisition date and time of the projection data by using the imaging date and time.

The reconstruction unit 64 generates the CT image by reconstructing the image based on the second output data corrected by the correction unit 62. The image based on the second output data is reconstructed by, for example, a filtered back projection method. The CT image including the plurality of tomographic images is an example of a radiation image.

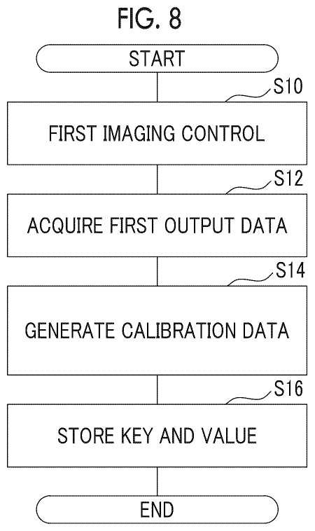

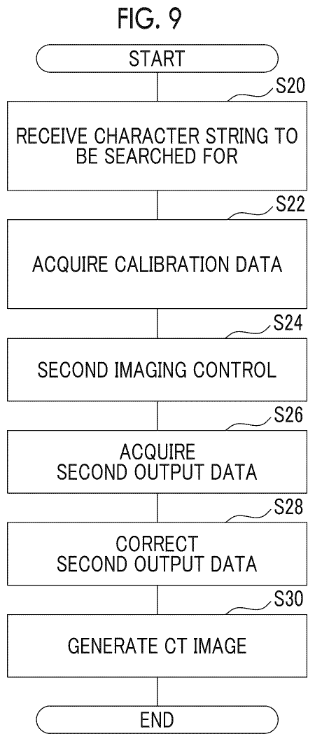

Next, an operation of the console 12 will be described with reference to FIGS. 8 and 9. The CPU 31 executes the information processing program 40 to execute a process of storing the calibration data shown in FIG. 8 and a process of generating the CT image shown in FIG. 9. The process of storing the calibration data is executed, for example, at a timing determined as a creation timing of the calibration data. The process of generating the CT image is executed, for example, in a case in which an instruction to start the execution is input by the user.

In step S10 of FIG. 8, the imaging controller 50 performs the first imaging control in accordance with the imaging conditions. In step S12, the acquisition unit 52 acquires the first output data obtained by the process of step S10 from the DAS 25. In step S14, the generation unit 54 generates the calibration data for correcting the error included in the output data of the radiation detector 22 based on the first output data acquired in step S12.

In step S16, the storage unit 56 includes, in the key, the character strings related to the imaging conditions for the CT image in the first imaging control executed in step S10, and includes, in the value, the calibration data of the radiation detector 22 generated based on the first output data acquired in step S12 in accordance with the imaging conditions. The storage unit 56 stores the key and the value in association with each other in the storage unit 33. In a case in which the process of step S16 ends, the process of storing the calibration data ends.

In step S20 of FIG. 9, the reception unit 58 receives the character string to be searched for. In step S22, the search unit 60 acquires the calibration data stored as the value corresponding to the key that partially matches the character string received in step S20 from the storage unit 33. In step S24, the imaging controller 50 performs the second imaging control in accordance with the imaging conditions.

In step S26, the acquisition unit 52 acquires the second output data obtained by the process of step S24 from the DAS 25. In step S28, the correction unit 62 corrects the second output data acquired in step S26 using the calibration data acquired in step S22. In step S30, the reconstruction unit 64 generates the CT image by reconstructing the image based on the second output data corrected by the process of step S28. In a case in which the process of step S30 ends, the process of generating the CT image ends.

As described above, according to the present embodiment, the time for the radiation detector to search for the calibration data can be shortened.

In the above-described embodiment, a case has been described in which the CT apparatus is applied as the radiographic imaging apparatus, but the technology of the present disclosure is not limited to this aspect. For example, as the radiographic imaging apparatus, an apparatus other than the CT apparatus, such as a radiographic imaging apparatus in which the radiation detector is built in a portable housing such as a digital radiography (DR) cassette, may be applied.

In addition, at least one of the functional units provided in the console 12 in the above-described embodiment may be provided in another device such as the control device provided in the gantry 18.

In addition, in the above-described embodiment, each process is executed by any computer. Any computer may execute these processes by a processor as hardware, a program as software, or a combination thereof. In such a case, the processor is configured to execute various processes in the above-described embodiment in cooperation with the program, and may function as each unit or each means in the above-described embodiment. In addition, the execution order of the processes by the processor is not limited to the above order, and may be changed as appropriate. Any computer may be a general-purpose computer, a dedicated computer, a workstation, or another system that can execute each process.

The processor may be configured by one or more kinds of hardware, and the type of hardware is not limited. For example, the processor may be configured by a programmable logic device such as a central processing unit (CPU), a microprocessing unit (MPU), or a field programmable gate array (FPGA), a dedicated circuit for executing specific processing, such as an application-specific integrated circuit (ASIC), or hardware such as a graphics processing unit (GPU) or a neural processing unit (NPU). Moreover, the type of hardware may be a combination of different kinds of hardware. In a case in which the plurality of types of hardware are configured to execute one or a plurality of processes of a certain processor, the plurality of types of hardware may be present in devices physically separated from each other or may be present in the same device. Furthermore, in any of the embodiments, the order of each process performed by the processor is not limited to the above-described order, and may be changed as appropriate. In addition, hardware is implemented in a form of an electrical circuit (circuitry) in which circuit elements, such as semiconductor elements, are combined.

Furthermore, the program may be software such as firmware or microcode. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

In addition, in the above-described embodiment, the aspect has been described in which the information processing program 40 is stored (installed) in the storage unit 33 in advance, but the present disclosure is not limited to this. The information processing program 40 may be provided in a form of being recorded in the recording medium such as a compact disc read only memory (CD-ROM), a digital versatile disc read only memory (DVD-ROM), and a universal serial bus (USB) memory. In addition, the information processing program 40 may be provided in a form being downloaded from an external device via a network. Further, the information processing program 40 can be provided as a program product. The program product includes products in any aspect for providing the program. For example, the program product includes a program provided through a network such as the Internet, and non-transitory computer-readable recording media such as a CD-ROM and a DVD in which the program is stored.

In regard to the above-described embodiment, the following supplementary notes are further disclosed.

### Supplementary Note 1

An information processing apparatus comprising: a processor, in which the information processing apparatus performs a process of storing data in a key-value format, and the processor is configured to: include, in a key, character strings related to imaging conditions for a radiation image; include, in a value, calibration data of a radiation detector provided in a radiographic imaging apparatus that captures the radiation image; and store the key and the value in association with each other in a storage device.

### Supplementary Note 2

The information processing apparatus according to supplementary note 1, in which the radiation image is a CT image, and the radiographic imaging apparatus is a CT apparatus.

### Supplementary Note 3

The information processing apparatus according to supplementary note 2, in which the CT apparatus includes a photon-counting radiation detector.

### Supplementary Note 4

The information processing apparatus according to supplementary note 2 or 3, in which the character strings related to the imaging conditions include a character string indicating whether or not a process of reducing the number of projection data items acquired by the CT apparatus is performed.

### Supplementary Note 5

The information processing apparatus according to any one of supplementary notes 1 to 4, in which the character strings related to the imaging conditions include a character string indicating a creation date and time of the calibration data.

### Supplementary Note 6

The information processing apparatus according to any one of supplementary notes 1 to 5, in which the processor is configured to: receive a character string to be searched for; and acquire the calibration data stored as the value corresponding to the key that partially matches the received character string from the storage device.

### Supplementary Note 7

The information processing apparatus according to any one of supplementary notes 1 to 6, in which the processor is configured to: during a period from a timing at which preparation for capturing the radiation image is started to a timing at which capturing the radiation image is started, acquire the calibration data corresponding to the imaging conditions for capturing the radiation image from the storage device.

### Supplementary Note 8

An information processing method executed by a processor provided in an information processing apparatus that performs a process of storing data in a key-value format, the information processing method comprising: including, in a key, character strings related to imaging conditions for a radiation image; including, in a value, calibration data of a radiation detector provided in a radiographic imaging apparatus that captures the radiation image; and storing the key and the value in association with each other in a storage device.

### Supplementary Note 9

An information processing program causing a processor provided in an information processing apparatus that performs a process of storing data in a key-value format, to execute a process comprising: including, in a key, character strings related to imaging conditions for a radiation image; including, in a value, calibration data of a radiation detector provided in a radiographic imaging apparatus that captures the radiation image; and storing the key and the value in association with each other in a storage device.

What is claimed is:

1. An information processing apparatus comprising:
a processor,
wherein the information processing apparatus performs a process of storing data in a key-value format, and
the processor is configured to:
include, in a key, character strings related to imaging conditions for a radiation image;
include, in a value, calibration data of a radiation detector provided in a radiographic imaging apparatus that captures the radiation image; and
store the key and the value in association with each other in a storage device.

2. The information processing apparatus according to claim 1,
wherein the radiation image is a CT image, and
the radiographic imaging apparatus is a CT apparatus.

3. The information processing apparatus according to claim 2,
wherein the CT apparatus includes a photon-counting radiation detector.

4. The information processing apparatus according to claim 2,
wherein the character strings related to the imaging conditions include a character string indicating whether or not a process of reducing the number of projection data items acquired by the CT apparatus is performed.

5. The information processing apparatus according to any claim 1,
wherein the character strings related to the imaging conditions include a character string indicating a creation date and time of the calibration data.

6. The information processing apparatus according to claim 1,
wherein the processor is configured to:
receive a character string to be searched for; and
acquire the calibration data stored as the value corresponding to the key that partially matches the received character string from the storage device.

7. The information processing apparatus according to claim 1,
wherein the processor is configured to:
during a period from a timing at which preparation for capturing the radiation image is started to a timing at which capturing the radiation image is started, acquire the calibration data corresponding to the imaging conditions for capturing the radiation image from the storage device.

8. An information processing method executed by a processor provided in an information processing apparatus that performs a process of storing data in a key-value format, the information processing method comprising:
including, in a key, character strings related to imaging conditions for a radiation image;
including, in a value, calibration data of a radiation detector provided in a radiographic imaging apparatus that captures the radiation image; and
storing the key and the value in association with each other in a storage device.

9. A non-transitory computer-readable storage medium storing an information processing program causing a processor provided in an information processing apparatus that performs a process of storing data in a key-value format, to execute a process comprising:
including, in a key, character strings related to imaging conditions for a radiation image;
including, in a value, calibration data of a radiation detector provided in a radiographic imaging apparatus that captures the radiation image; and
storing the key and the value in association with each other in a storage device.
